(19) United States
(12) Patent Application Publication
(10) Pub. No.: US 2008/0014221 A1
(43) Pub. Date: Jan. 17, 2008

(54) Mycoplasma hyopneumoniae vaccine

(76) Inventors: Gregory Charles Royer, Fulton, MO (US); Russell Howser, Fulton, MO (US); Catherine Charreyre, Saint Laurent De Mure (FR); Francis William Milward, Bogart, GA (US)

Correspondence Address: Judy Jarecki-Black, Ph.D., J.D., 3239 Satellite Blvd., Duluth, GA, 30096, US

(21) Appl. No.: 11/711,358

(22) Filed: Feb. 27, 2007

Related U.S. Application Data
(60) Provisional application No. 60/778,987, filed on Mar. 3, 2006.

(51) Int. Cl. A61K 39/04

(52) U.S. Cl. 424/264.1

(57) ABSTRACT

The invention provides a novel method of vaccination of a pig against Mycoplasma hyopneumoniae by administering to a weaned piglet or a sow, a single dose of an effective amount of a Mycoplasma hyopneumoniae vaccine. The Mycoplasma hyopneumoniae vaccine can be a whole or partial cell inactivated or modified live preparation, a subunit vaccine, or a nucleic acid or DNA vaccine.

### INCORPORATION BY REFERENCE

This application claims benefit of U.S. provisional patent application Ser. No. 60/778,987 filed Mar. 3, 2006.

Reference is made to U.S. patent application Ser. Nos. 10/899,181 filed Jul. 26, 2004, U.S. Ser. No. 09/232,279 filed Jan. 15, 1999, now U.S. Pat. No. 6,376,473 issued Apr. 23, 2002, U.S. Ser. No. 09/232,469 filed Jan. 15, 1999 now U.S. Pat. No. 6,451,770 issued Sep. 17, 2002, U.S. Ser. No. 09/232,477 filed Jan. 15, 1999, now U.S. Pat. No. 6,228,846, issued May 8, 2001, U.S. Ser. No. 09/232,478 filed Jan. 15, 1999 now U.S. Pat. No. 6,207,166 issued Mar. 27, 2001, U.S. Ser. No. 09/232,479 filed Jan. 15, 1999, now U.S. Pat. No. 6,221,362 issued Apr. 24, 2001, U.S. Ser. No. 09/347,594 filed Jul. 1, 1999 now U.S. Pat. No. 6,217,883 issued Apr. 17, 2001, U.S. Ser. No. 09/583,350 filed May 31, 2000 now U.S. Pat. No. 6,517,843 issued Feb. 11, 2003, U.S. Ser. No. 09/616,781 filed Jul. 14, 2000 now U.S. Pat. No. 6,534,066 issued Mar. 18, 2003, U.S. Ser. No. 09/680,228 filed Oct. 6, 2000, U.S. Ser. No. 09/784,962 filed Feb. 16, 2001, U.S. Ser. No. 09/784,982 filed Feb. 16, 2001, now U.S. Pat. No. 6,586,412, issued Jul. 1, 2003, U.S. Ser. No. 09/784,990 filed Feb. 16, 2001, now U.S. Pat. No. 6,464,984 issued Oct. 15, 2002, U.S. Ser. No. 09/785,055 filed Feb. 16, 2001 now U.S. Pat. No. 6,558,674 issued May 6, 2003, U.S. Ser. No. 10/077,489 filed Feb. 15, 2002 now U.S. Pat. No. 6,803,361 issued Oct. 12, 2004, U.S. Ser. No. 10/085,519 filed Feb. 28, 2002, now U.S. Pat. No. 6,818,628 issued Nov. 16, 2004, U.S. Ser. No. 10/211,502 filed Aug. 2, 2002, U.S. Ser. No. 10/229,412 filed Aug. 28, 2002, U.S. Ser. No. 10/238,114 filed Sep. 10, 2002, U.S. Ser. No. 10/368,861 filed Feb. 18, 2003, U.S. Ser. No. 10/391,498 filed Mar. 18, 2003, U.S. Ser. No. 10/406,686 filed Apr. 4, 2003, U.S. Ser. No. 10/730,206 filed Dec. 8, 2003, now U.S. Pat. No. 6,908,620 issued Jun. 21, 2005, U.S. Ser. No. 10/838,122 filed May 3, 2004, U.S. Ser. No. 10/983,928 filed Nov. 8, 2004, U.S. Ser. No. 11,038,682 filed Jan. 19, 2005, U.S. Ser. No. 11/079,559 filed Mar. 14, 2005, U.S. Ser. No. 11/106,780 filed Apr. 15, 2005, now U.S. Pat. No. 6,998,127 issued Feb. 14, 2006, U.S. Ser. No. 11/156,829 filed Jun. 20, 2005, U.S. Ser. No. 11/184,236 filed Jul. 19, 2005, U.S. Ser. No. 11/196,722 filed Aug. 3, 2005, U.S. Ser. No. 11/211,983 filed Sep. 25, 2005, U.S. Ser. No. 11/348,084 filed Feb. 6, 2006, U.S. Ser. No. 60/151,564 filed Aug. 31, 1999, U.S. Ser. No. 60/318,686 filed Sep. 12, 2001, U.S. Ser. No. 60/366,014 filed Mar. 20, 2002, U.S. Ser. No. 60/370,282 filed Apr. 5, 2002, U.S. Ser. No. 60/432,298 filed Dec. 9, 2002, U.S. Ser. No. 60/490,345 filed Jul. 24, 2003, USSN 60/519,571 filed Nov. 13, 2003, U.S. Ser. No. 60/522,636 filed Mar. 12, 2004, U.S. Ser. No. 60/576,771 filed Jun. 4, 2004, U.S. Ser. No. 60/581,689 filed Jun. 15, 2004, U.S. Ser. No. 60/581,698 filed Jun. 21, 2004, U.S. Ser. No. 60/627,878 filed Nov. 15, 2004, U.S. Ser. No. 60/662,646 filed Mar. 17, 2005, and U.S. Ser. No. 60/736,452 filed Nov. 14, 2005.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

The invention provides a Mycoplasma hyopneumoniae vaccine and methods for administering the same.

### BACKGROUND OF THE INVENTION

Mycoplasma hyopneumoniae, the cause of enzootic pneumonia, remains an important pathogen in the swine industry. This small, complex organism colonizes the ciliated cells of the respiratory tract, resulting in little exposure to the immune system. The lung lesions, generally observed in young pigs, are characterised by a hyperplasia of the epithelial cells and an increased perivascular and peribronchiolar accumulation of mononuclear cells. Following M. hyopneumoniae infection, immune reactions are observed and resistance is induced in pigs. (reviewed in, e.g., Thacker, Anim Health Res Rev. 2004 December;5(2):317-20 and Kobisch & Friis, Rev Sci Tech. 1996 December;15(4):1569-605). Clinical symptoms and lesion development are the result of the pathogenic capacity of M. hyopneumoniae and the defense reactions in the lung. The economic relevance of pneumonia is influenced to a large extent by common secondary infections which follow an initial M. hyopneumoniae infection. Different tests for the diagnosis of pneumonia in individual pigs and in groups are available. Treatment and control is not simple since enzootic pneumonia is a multi-factorial disease (reviewed in, e.g., Maes et al., Vet Q. 1996 September;18(3):104-9).

M. hyopneumoniae is also associated with porcine respiratory disease complex (PRDC), a multifactorial respiratory syndrome that includes several respiratory pathogens. The pathogens most commonly isolated from pigs with clinical signs of PRDC either infect the cells of the immune system or induce significant immunopathology. Thus, PRRSV and M. hyopneumoniae, the two most common pathogens associated with PRDC, alter the ability of the respiratory immune system to respond to their presence and the presence of other pathogens. By changing the respiratory immune system, these two common pathogens increase the susceptibility to the many other pathogens associated with PRDC (reviewed in, e.g., Thacker, Vet Clin North Am Food Anim Pract. 2001 November;17(3):551-65).

The majority of known vaccines against M. hyopneumoniae have been based on adjuvanted inactivated whole cell preparations of M. hyopneumoniae. Commercial sources include RESPIFEND (Fort Dodge, American Home Products), HYORESP (Merial Ltd), M+PAC (Schering Plough), PROSYSTEM M (Intervet), INGLEVAC M (Boehringer), RESPISURE (Pfizer Inc.), and STELLAMUNE MYCOPLASMA (Pfizer Inc.). WO 03/003941 relates to a needle administered composition.

There remains a need for an effective M. hyopneumoniae vaccine effective in a single dose treatment that is easy to be administered to a large number of animals and which is cost effective. The prevalence of several infectious diseases of swine, such as mycoplasmosis, over the past several years have made it necessary to develop vaccines and accompanying vaccination schedules. These schedules include the vaccination of pigs prior to maturity, which present logistical difficulties, i.e. the high number of pigs to vaccinate, and that vaccination, via needle injection, is made either while holding the animals, which is labour intensive, or without holding them, which prevents certainty as to the effectiveness of the administration of the inoculation. Such a vaccine would eliminate the need for multiple dosing and thereby significantly decrease the costs and labor associated with the worldwide massive vaccination of swine herds.

Citation or identification of any document in this application is not an admission that such document is available as prior art to the present invention.

### SUMMARY OF THE INVENTION

The present invention provides a method of treating or preventing a disease or disorder in an animal, advantageously a pig, caused by infection with Mycoplasma hyopneumoniae which may comprise administering to an animal, advantageously a pig, an effective amount of a single dose of a Mycoplasma hyopneumoniae vaccine.

The M. hyopneumoniae vaccine may comprise a whole or partial cell preparation, such as a bacterin or modified live preparation, a subunit vaccine, such as a subunit vaccine which may comprise one or more M. hyopneumoniae derived polypeptides or proteins, immunogenic fragments of such polypeptides or proteins, or one or more M. hyopneumoniae genes encoding such proteins, polypeptides or immunogenic fragments. Advantageously, the M. hyopneumoniae vaccine is an inactivated vaccine. In another advantageous embodiment, the M. hyopneumoniae vaccine may further comprise an adjuvant.

Another of the present invention is a vaccination method against M. hyopneumoniae, which may comprise the step of administration to an animal, advantageously a pig, an efficient amount of a M. hyopneumoniae vaccine using a liquid jet needle-free injector, which administration elicits a safe and protective immune response against M. hyopneumoniae. Another object is a vaccination kit or set, which may comprise such a liquid jet needle-free injector and at least one vaccine vial containing a M. hyopneumoniae vaccine, operatively assembled to perform the administration of the vaccine to an animal, advantageously a pig, and to elicit a safe and protective immune response against M. hyopneumoniae.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### DETAILED DESCRIPTION

The present invention provides a method of treating or preventing a disease or disorder in an animal, advantageously a pig, caused by infection with Mycoplasma hyopneumoniae which may comprise administering to an animal, advantageously a pig, an effective amount of a single dose of a Mycoplasma hyopneumoniae vaccine.

As used herein, the term “animal” includes all vertebrate animals including humans. It also includes an individual animal in all stages of development, including embryonic and fetal stages. In particular, the term “vertebrate animal” includes, but not limited to, humans, canines (e.g., dogs), felines (e.g., cats); equines (e.g., horses), bovines (e.g., cattle) porcine (e.g., pigs), as well as in avians. The term “avian” as used herein refers to any species or subspecies of the taxonomic class ava, such as, but not limited to, chickens (breeders, broilers and layers), turkeys, ducks, a goose, a quail, pheasants, parrots, finches, hawks, crows and ratites including ostrich, emu and cassowary.

As used herein, the term “pig” refers to an animal of porcine origin. The term “boar” refers to an entire male pig over six months of age destined as a sire. The term “gilt” refers to a young female pig who has not produced first litter up to first farrowing. The term “hog” refers to a castrated male pig. The term “piglet” refers to a young pig. The term “porker” refers to a breed of pig breed for good pork meat cuts. The term “stores” refers to a pig which may be about 10-12 weeks old. The term “sow” refers to a female of reproductive age and capability or a female pig after she has had her first litter. The term “weaned piglet” or “weaner” refers to a young pig which may be about 11 to about 24 days of age, about two to three weeks of age, about three to five weeks of age or about five to eight weeks old weeks of age.

As used herein, the term “virulent” means an isolate that retains its ability to be infectious in an animal host.

As used herein, the term “inactivated vaccine” means a vaccine composition containing an infectious organism or pathogen that is no longer capable of replication or growth. The pathogen may be bacterial, viral, protozoal or fungal in origin. Inactivation may be accomplished by a variety of methods including freeze-thawing, chemical treatment (for example, treatment with thimerosal or formalin), sonication, radiation, heat or any other convention means sufficient to prevent replication or growth of the organism while maintaining its immunogenicity.

As used herein, the term “immune response” refers to a response elicited in an animal. An immune response may refer to cellular immunity (CMI); humoral immunity or may involve both. The present invention also contemplates a response limited to a part of the immune system. For example, a vaccine composition of the present invention may specifically induce an increased gamma interferon response.

As used herein, the term “antigen” or “immunogen” means a substance that induces a specific immune response in a host animal. The antigen may comprise a whole organism, killed, attenuated or live; a subunit or portion of an organism; a recombinant vector containing a polynucleotide encoding an immunogen, capable of inducing an immune response upon presentation to a host animal; a protein, a polypeptide, a peptide, an epitope, a hapten, or any combination thereof.

As used herein, the term “multivalent” means a vaccine containing more than one antigen from different genera or species of microorganisms (for example, a vaccine comprising antigens from Pasteurella multocida, Salmonella, Escherichia coli, Haemophilus somnus and Clostridium).

As used herein, the term “adjuvant” means a substance added to a vaccine to increase a vaccine's immunogenicity. The mechanism of how an adjuvant operates is not entirely known.

Some adjuvants are believed to enhance the immune response by slowly releasing the antigen, while other adjuvants present the immunogen to the host immune system more efficiently or effectively or stimulate the production of specific cytokines.

As used herein, the terms “pharmaceutically acceptable carrier” and “pharmaceutically acceptable vehicle” are interchangeable and refer to a fluid vehicle for containing vaccine antigens that can be injected into a host without adverse effects. Suitable pharmaceutically acceptable carriers known in the art include, but are not limited to, sterile water, saline, glucose, dextrose, or buffered solutions. Carriers may include auxiliary agents including, but not limited to, diluents, stabilizers (i.e., sugars and amino acids), preservatives, wetting agents, emulsifying agents, pH buffering agents, viscosity enhancing additives, colors and the like.

As used herein, the term “vaccine composition” includes at least one antigen or immunogen in a pharmaceutically acceptable vehicle useful for inducing an immune response in a host. Vaccine compositions can be administered in dosages and by techniques well known to those skilled in the medical or veterinary arts, taking into consideration such factors as the age, sex, weight, species and condition of the recipient animal, and the route of administration. The route of administration can be percutaneous e;g. intradermal, intramuscular, subcutaneous. Vaccine compositions can be administered alone, or can be co-administered or sequentially administered with other treatments or therapies. The compositions can contain auxiliary substances such as wetting or emulsifying agents, pH buffering agents, adjuvants, or viscosity enhancing additives, preservatives, colors, and the like, depending upon the route of administration and the preparation desired. Standard pharmaceutical texts, such as “Remington's Pharmaceutical Sciences,” 1990 may be consulted to prepare suitable preparations, without undue experimentation.

The M. hyopneumoniae vaccine may comprise a whole or partial cell preparation and/or the supernatant, such as a bacterin or modified live preparation, a subunit vaccine, such as a subunit vaccine which may comprise one or more M. hyopneumoniae derived polypeptides or proteins, immunogenic fragments of such polypeptides or proteins, or one or more M. hyopneumoniae genes encoding such proteins, polypeptides or immunogenic fragments which genes or nucleic acids. Advantageously, the M. hyopneumoniae vaccine is an inactivated vaccine. In another advantageous embodiment, the M. hyopneumoniae vaccine may further comprise an adjuvant.

To obtain an inactivated immunologic, or vaccine composition, the pathogen is produced in a medium capable of supporting the growth of Mycoplasma. The selected culture medium may include media known to those of skill in the art to propagate Mycoplasma such as described in R. Ross et al Am. J. Vet. Res. 1984, 45, 1899-1905, B. Kristensen et al Am. J. Vet. Res. 1981, 42, 784-788, WO 91/18627, U.S. Pat. No. 5,338,543 or other similar references. The Mycoplasma is preferably inactivated after harvesting and, optionally, subjected to clarification by means of a chemical treatment using, for example, formalin or formaldehyde, beta-propiolactone, ethyleneimine, binary ethyleneimine (BEI), thimerosal, and the like, and/or a physical treatment (e.g. a heat treatment or sonication). Methods for inactivation are well known to those of skill in the art. Mycoplasma hyopneumoniae bacterium may be inactivated by formaldehyde treatment (Ross R. F. et al., Am. J. Vet. Res., 1984, 45: 1899-1905), by ethylenimine or BEI treatment (see, e.g., WO 91/18627), or by thimerosal treatment (U.S. Pat. Nos. 5,968,525 and 5,338,543). The inactivating agent can be neutralized or removed by a purification step.

The inactivated pathogen can be concentrated by conventional concentration techniques, in particular by ultrafiltration, and/or purified by conventional purification means, in particular using chromatography techniques including, but not limited to, gel-filtration or by ultrafiltration. As used herein, the term “immunogenicity” means capable of producing an immune response in a host animal against an antigen or antigens. This immune response forms the basis of the protective immunity elicited by a vaccine against a specific infectious organism.

The method of the present invention can be practiced using subunit vaccines having purified M. hyopneumoniae immunogenic proteins, polypeptides and immunogenic fragments of such proteins and polypeptides. Such proteins and polypeptides can be prepared using techniques known in the art. Further, methods which are well known to those skilled in the art can be used to determine protein purity or homogeneity, such as polyacrylamide gel electrophoresis of a sample, followed by visualizing a single polypeptide band on a staining gel. Higher resolution may be determined using HPLC or other similar methods well known in the art. In a specific embodiment, the vaccine used in the present invention comprises at least one protein of M. hyopneumoniae such as, but not limited to, P46, P65, P97, P102, P70, P50 and P44. For a sequence of the M. hyopneumoniae genome, reference is made to Minion et al., J Bacteriol. 2004 November;186(21):7123-33.

In other embodiments, the vaccine used in the method of the present invention comprises a M. hyopneumoniae bacterin (inactivated whole or partial cell or modified live) or a M. hyopneumoniae protein or polypeptide or immunogenic fragment thereof and at least one other immunogen (inactivated whole or partial cell or modified live) or an immunogenic or antigenic protein, polypeptide or immunogenic fragment thereof, and is preferably a viral, bacterial or parasitic polypeptide. In a further specific embodiment, the immunogenic fragments of such proteins or polypeptides have a sequence comprising at least 10, at least 15, at least 20, at least 25, at least 30, at least 35, at least 40, at least 45, at least 50, at least 55, at least 60, at least 65, at least 70, at least 75, at least 80, at least 85, at least 90, at least 95 or at least 100 contiguous amino acids of the immunogenic proteins and polypeptides used in the method of the present invention, including but not limited to P46, P65, P97, P102, P70, P50 and P44.

Further, the M. hyopneumoniae proteins for use in vaccines are substantially pure or homogeneous. The method of the present invention uses proteins or polypeptides which are typically purified from host cells expressing recombinant nucleotide sequences encoding these proteins. Such protein purification can be accomplished by a variety of methods well known in the art. See, for example, the techniques described in “Methods In Enzymology”, 1990, Academic Press, Inc., San Diego, “Protein Purification: Principles and practice”, 1982, Springer-Verlag, New York.

The invention further encompasses at least one M. hyopneumoniae immunogen contained in a vector molecule or an expression vector and operably linked to a promoter element and optionally to an enhancer.

In an advantageous embodiment, the promoter is the promoter of the cytomegalovirus (CMV) immediate early gene In another embodiment, the enhancers and/or promoters include various cell or tissue specific promoters (e.g., muscle, endothelial cell, liver, somatic cell or stem cell), various viral promoters and enhancers. Examples of muscle-specific promoters and enhancers have been described are known to one of skill in the art (see, e.g., Li et al., Gene Ther. December 1999;6(12):2005-11; Li et al., Nat Biotechnol. March 1999;17(3):241-5 and Loirat et al., Virology. Jul. 20, 1999;260(1):74-83; the disclosures of which are incorporated by reference in their entireties).

Promoters and enhancers that may be employed in the present invention include, but are not limited to LTR or the Rous sarcoma virus, TK of HSV-1, early or late promoter of SV40, adenovirus major late (MLP), phosphoglycerate kinase, metallothionein, alpha-1 antitrypsin, albumin, collagenese, elastase I, beta-actin, beta-globin, gamma-globin, alpha-fetoprotein, muscle creatin kinase.

A “vector” refers to a recombinant DNA or RNA plasmid or virus that comprises a heterologous polynucleotide to be delivered to a target cell, either in vitro or in vivo. The heterologous polynucleotide may comprise a sequence of interest for purposes of therapy, and may optionally be in the form of an expression cassette. As used herein, a vector need not be capable of replication in the ultimate target cell or subject. The term includes cloning vectors also included are viral vectors.

The term “recombinant” means a polynucleotide semisynthetic, or synthetic origin which either does not occur in nature or is linked to another polynucleotide in an arrangement not found in nature.

“Heterologous” means derived from a genetically distinct entity from the rest of the entity to which it is being compared. For example, a polynucleotide, may be placed by genetic engineering techniques into a plasmid or vector derived from a different source, and is a heterologous polynucleotide. A promoter removed from its native coding sequence and operatively linked to a coding sequence other than the native sequence is a heterologous promoter.

The polynucleotides of the invention may comprise additional sequences, such as additional encoding sequences within the same transcription unit, controlling elements such as promoters, ribosome binding sites, polyadenylation sites, additional transcription units under control of the same or a different promoter, sequences that permit cloning, expression, homologous recombination, and transformation of a host cell, and any such construct as may be desirable to provide embodiments of this invention.

The present invention encompasses a vector expressing M. hyopneumoniae immunogen or variants or analogues or fragments. Elements for the expression of M. hyopneumoniae immunogen are advantageously present in an inventive vector. At a minimum, this comprises, consists essentially of, or consists of an initiation codon (ATG), a stop codon and a promoter, and optionally also a polyadenylation sequence for certain vectors such as plasmid and certain viral vectors, e.g., viral vectors other than poxviruses. When the polynucleotide encodes a polyprotein fragment, e.g. a M. hyopneumoniae immunogen, advantageously, in the vector, an ATG is placed at 5′ of the reading frame and a stop codon is placed at 3′. Other elements for controlling expression may be present, such as enhancer sequences, stabilizing sequences, such as intron and signal sequences permitting the secretion of the protein.

Methods for making and/or administering a vector or recombinants or plasmid for expression of gene products of genes of the invention either in vivo or in vitro can be any desired method, e.g., a method which is by or analogous to the methods disclosed in, or disclosed in documents cited in: U.S. Pat. Nos. 4,603,112; 4,769,330; 4,394,448; 4,722,848; 4,745,051; 4,769,331; 4,945,050; 5,494,807; 5,514,375; 5,744,140; 5,744,141; 5,756,103; 5,762,938; 5,766,599; 5,990,091; 5,174,993; 5,505,941; 5,338,683; 5,494,807; 5,591,639; 5,589,466; 5,677,178; 5,591,439; 5,552,143; 5,580,859; 6,130,066; 6,004,777; 6,130,066; 6,497,883; 6,464,984; 6,451,770; 6,391,314; 6,387,376; 6,376,473; 6,368,603; 6,348,196; 6,306,400; 6,228,846; 6,221,362; 6,217,883; 6,207,166; 6,207,165; 6,159,477; 6,153,199; 6,090,393; 6,074,649; 6,045,803; 6,033,670; 6,485,729; 6,103,526; 6,224,882; 6,312,682; 6,348,450 and 6,312,683; U.S. patent application Ser. No. 920,197, filed Oct. 16, 1986; WO 90/01543; WO 91/11525; WO 94/16716; WO 96/39491; WO 98/33510; EP 265785; EP 0 370 573; Andreansky et al., Proc. Natl. Acad. Sci. USA 1996;93:11313-11318; Ballay et al., EMBO J. 1993;4:3861-65; Felgner et al., J. Biol. Chem. 1994;269:2550-2561; Frolov et al., Proc. Natl. Acad. Sci. USA 1996;93:11371-11377; Graham, Tibtech 1990;8:85-87; Grunhaus et al., Sem. Virol. 1992;3:237-52; Ju et al., Diabetologia 1998;41:736-739; Kitson et al., J. Virol. 1991;65:3068-3075; McClements et al., Proc. Natl. Acad. Sci. USA 1996;93:11414-11420; Moss, Proc. Natl. Acad. Sci. USA 1996;93:11341-11348; Paoletti, Proc. Natl. Acad. Sci. USA 1996;93:11349-11353; Pennock et al., Mol. Cell. Biol. 1984;4:399406; Richardson (Ed), Methods in Molecular Biology 1995;39, “Baculovirus Expression Protocols,” Humana Press Inc.; Smith et al. (1983) Mol. Cell. Biol. 1983;3:2156-2165; Robertson et al., Proc. Natl. Acad. Sci. USA 1996;93:11334-11340; Robinson et al., Sem. Immunol. 1997;9:271; and Roizman, Proc. Natl. Acad. Sci. USA 1996;93:11307-11312. Thus, the vector in the invention can be any suitable recombinant virus or virus vector, such as a poxvirus (e.g., vaccinia virus, avipox virus, canarypox virus, fowlpox virus, raccoonpox virus, swinepox virus, etc.), adenovirus (e.g., human adenovirus, canine adenovirus), herpesvirus (e.g. canine herpesvirus), baculovirus, retrovirus, etc. (as in documents incorporated herein by reference); or the vector can be a plasmid. The herein cited and incorporated herein by reference documents, in addition to providing examples of vectors useful in the practice of the invention, can also provide sources for non-M. hyopneumoniae immunogens, e.g., non-M. hyopneumoniae immunogens, non-M. hyopneumoniae immunogens peptides or fragments thereof, cytokines, etc. to be expressed by vector or vectors in, or included in, the compositions of the invention.

The present invention also relates to preparations comprising vectors, such as expression vectors, e.g., therapeutic compositions. The preparations can comprise, consist essentially of, or consist of one or more vectors, e.g., expression vectors, such as in vivo expression vectors, comprising, consisting essentially or consisting of (and advantageously expressing) one or more of M. hyopneumoniae immunogens. Advantageously, the vector contains and expresses a polynucleotide that includes, consists essentially of, or consists of a coding region encoding one or more M. hyopneumoniae immunogens a pharmaceutically or veterinarily acceptable carrier, excipient or vehicle. Thus, according to an embodiment of the invention, the other vector or vectors in the preparation comprises, consists essentially of or consists of a polynucleotide that encodes, and under appropriate circumstances the vector expresses one or more other proteins of a M. hyopneumoniae immunogen or a fragment thereof.

According to another embodiment, the vector or vectors in the preparation comprise, or consist essentially of, or consist of polynucleotide(s) encoding one or more proteins or fragment(s) thereof of a M. hyopneumoniae immunogen, the vector or vectors have expression of the polynucleotide(s). The inventive preparation advantageously comprises, consists essentially of, or consists of, at least two vectors comprising, consisting essentially of, or consisting of, and advantageously also expressing, advantageously in vivo under appropriate conditions or suitable conditions or in a suitable host cell, polynucleotides from different M. hyopneumoniae isolates encoding the same proteins and/or for different proteins, but advantageously for the same proteins. Preparations containing one or more vectors containing, consisting essentially of or consisting of polynucleotides encoding, and advantageously expressing, advantageously in vivo, M. hyopneumoniae peptide, fusion protein or an epitope thereof.

According to one embodiment of the invention, the expression vector is a viral vector, in particular an in vivo expression vector. In an advantageous embodiment, the expression vector is an adenovirus vector. Advantageously, the adenovirus is a human Ad5 vector, an E1-deleted and/or an E3-deleted adenovirus.

In one particular embodiment the viral vector is a poxvirus, e.g. a vaccinia virus or an attenuated vaccinia virus, (for instance, MVA, a modified Ankara strain obtained after more than 570 passages of the Ankara vaccine strain on chicken embryo fibroblasts; see Stickl & Hochstein-Mintzel, Munch. Med. Wschr., 1971, 113, 1149-1153; Sutter et al., Proc. Natl. Acad. Sci. U.S.A., 1992, 89, 10847-10851; available as ATCC VR-1508; or NYVAC, see U.S. Pat. No. 5,494,807, for instance, Examples 1 to 6 and et seq of U.S. Pat. No. 5,494,807 which discuss the construction of NYVAC, as well as variations of NYVAC with additional ORFs deleted from the Copenhagen strain vaccinia virus genome, as well as the insertion of heterologous coding nucleic acid molecules into sites of this recombinant, and also, the use of matched promoters; see also WO96/40241), an avipox virus or an attenuated avipox virus (e.g., canarypox, fowlpox, dovepox, pigeonpox, quailpox, ALVAC or TROVAC; see, e.g., U.S. Pat. Nos. 5,505,941, 5,494,807), swinepox, raccoonpox, camelpox, or myxomatosis virus.

According to another embodiment of the invention, the poxvirus vector is a canarypox virus or a fowlpox virus vector, advantageously an attenuated canarypox virus or fowlpox virus. In this regard, is made to the canarypox available from the ATCC under access number VR-111. Attenuated canarypox viruses are described in U.S. Pat. No. 5,756,103 (ALVAC) and WO01/05934. Numerous fowlpox virus vaccination strains are also available, e.g. the DIFTOSEC CT strain marketed by MERIAL and the NOBILIS VARIOLE vaccine marketed by INTERVET; and, reference is also made to U.S. Pat. No. 5,766,599 which pertains to the atenuated fowlpox strain TROVAC.

For information on the method to generate recombinants thereof and how to administer recombinants thereof, the skilled artisan can refer documents cited herein and to WO90/12882, e.g., as to vaccinia virus mention is made of U.S. Pat. Nos. 4,769,330, 4,722,848, 4,603,112, 5,110,587, 5,494,807, and 5,762,938 inter alia; as to fowlpox, mention is made of U.S. Pat. Nos. 5,174,993, 5,505,941 and U.S. Pat. No. 5,766,599 inter alia; as to canarypox mention is made of U.S. Pat. No. 5,756,103 inter alia; as to swinepox mention is made of U.S. Pat. No. 5,382,425 inter alia; and, as to raccoonpox, mention is made of WO00/03030 inter alia.

When the expression vector is a vaccinia virus, insertion site or sites for the polynucleotide or polynucleotides to be expressed are advantageously at the thymidine kinase (TK) gene or insertion site, the hemagglutinin (HA) gene or insertion site, the region encoding the inclusion body of the A type (ATI); see also documents cited herein, especially those pertaining to vaccinia virus. In the case of canarypox, advantageously the insertion site or sites are ORF(s) C3, C5 and/or C6; see also documents cited herein, especially those pertaining to canarypox virus. In the case of fowlpox, advantageously the insertion site or sites are ORFs F7 and/or F8; see also documents cited herein, especially those pertaining to fowlpox virus. The insertion site or sites for MVA virus area advantageously as in various publications, including Carroll M. W. et al., Vaccine, 1997, 15 (4), 387-394; Stittelaar K. J. et al., J. Virol., 2000, 74 (9), 4236-4243; Sutter G. et al., 1994, Vaccine, 12 (11), 1032-1040; and, in this regard it is also noted that the complete MVA genome is described in Antoine G., Virology, 1998, 244, 365-396, which enables the skilled artisan to use other insertion sites or other promoters.

Advantageously, the polynucleotide to be expressed is inserted under the control of a specific poxvirus promoter, e.g., the vaccinia promoter 7.5 kDa (Cochran et al., J. Virology, 1985, 54, 30-35), the vaccinia promoter 13L (Riviere et al., J. Virology, 1992, 66, 3424-3434), the vaccinia promoter HA (Shida, Virology, 1986, 150, 451-457), the cowpox promoter ATI (Funahashi et al., J. Gen. Virol., 1988, 69, 35-47), the vaccinia promoter H6 (Taylor J. et al., Vaccine, 1988, 6, 504-508; Guo P. et al. J. Virol., 1989, 63, 4189-4198; Perkus M. et al., J. Virol., 1989, 63, 3829-3836), inter alia.

In a particular embodiment the viral vector is an adenovirus, such as a human adenovirus (HAV) or a canine adenovirus (CAV).

In one embodiment the viral vector is a human adenovirus, in particular a serotype 5 adenovirus, rendered incompetent for replication by a deletion in the E1 region of the viral genome, in particular from about nucleotide 459 to about nucleotide 3510 by reference to the sequence of the hAdS disclosed in Genbank under the accession number M73260 and in the referenced publication J. Chroboczek et al Virol. 1992, 186, 280-285. The deleted adenovirus is propagated in E1-expressing 293 (F. Graham et al J. Gen. Virol. 1977, 36, 59-72) or PER cells, in particular PER.C6 (F. Falloux et al Human Gene Therapy 1998, 9, 1909-1917). The human adenovirus can be deleted in the E3 region, in particular from about nucleotide 28592 to about nucleotide 30470. The deletion in the E1 region can be done in combination with a deletion in the E3 region (see, e.g. J. Shriver et al. Nature, 2002, 415, 331-335, F. Graham et al Methods in Molecular Biology Vol. 7: Gene Transfer and Expression Protocols Edited by E. Murray, The Human Press Inc, 1991, p 109-128; Y. Ilan et al Proc. Natl. Acad. Sci. 1997, 94, 2587-2592; U.S. Pat. No. 6,133,028; U.S. Pat. No. 6,692,956; S. Tripathy et al Proc. Natl. Acad. Sci. 1994, 91, 11557-11561; B. Tapnell Adv. Drug Deliv. Rev. 1993, 12, 185-199; X. Danthinne et al Gene Thrapy 2000, 7, 1707-1714; K. Berkner Bio Techniques 1988, 6, 616-629; K. Berkner et al Nucl. Acid Res. 1983, 11, 6003-6020; C. Chavier et al J. Virol. 1996, 70, 4805-4810). The insertion sites can be the E1 and/or E3 loci (region) eventually after a partial or complete deletion of the E1 and/or E3 regions. Advantageously, when the expression vector is an adenovirus, the polynucleotide to be expressed is inserted under the control of a promoter functional in eukaryotic cells, such as a strong promoter, preferably a cytomegalovirus immediate-early gene promoter (CMV-IE promoter), in particular the enhancer/promoter region from about nucleotide −734 to about nucleotide +7 in M. Boshart et al Cell 1985, 41, 521-530 or the enhancer/promoter region from the pCI vector from Promega Corp. The CMV-IE promoter is advantageously of murine or human origin. The promoter of the elongation factor 1.alpha. can also be used. In one particular embodiment a promoter regulated by hypoxia, e.g. the promoter HRE described in K. Boast et al Human Gene Therapy 1999, 13, 2197-2208), can be used. A muscle specific promoter can also be used (X. Li et al Nat. Biotechnol. 1999, 17, 241-245). Strong promoters are also discussed herein in relation to plasmid vectors. In one embodiment, a splicing sequence can be located downstream of the enhancer/promoter region. For example, the intron 1 isolated from the CMV-IE gene (R. Stenberg et al J. Virol. 1984, 49, 190), the intron isolated from the rabbit or human .beta.-globin gene, in particular the intron 2 from the b-globin gene, the intron isolated from the immunoglobulin gene, a splicing sequence from the SV40 early gene or the chimeric intron sequence isolated from the pCI vector from Promege Corp. comprising the human .beta.-globin donor sequence fused to the mouse immunoglobulin acceptor sequence (from about nucleotide 890 to about nucleotide 1022 in Genbank under the accession number CVU47120). A poly(A) sequence and terminator sequence can be inserted downstream the polynucleotide to be expressed, e.g. a bovine growth hormone gene, in particular from about nucleotide 2339 to about nucleotide 2550 in Genbank under the accession number BOVGHRH, a rabbit .beta.-globin gene or a SV40 late gene polyadenylation signal.

In another embodiment the viral vector is a canine adenovirus, in particular a CAV-2 (see, e.g. L. Fischer et al. Vaccine, 2002, 20, 3485-3497; U.S. Pat. No. 5,529,780; U.S. Pat. No. 5,688,920; PCT Application No. WO95/14102). For CAV, the insertion sites can be in the E3 region and /or in the region located between the E4 region and the right ITR region (see U.S. Pat. No. 6,090,393; U.S. Pat. No. 6,156,567). In one embodiment the insert is under the control of a promoter, such as a cytomegalovirus immediate-early gene promoter (CMV-IE promoter) or a promoter already described for a human adenovirus vector. A poly(A) sequence and terminator sequence can be inserted downstream the polynucleotide to be expressed, e.g. a bovine growth hormone gene or a rabbit .beta.-globin gene polyadenylation signal.

In another particular embodiment the viral vector is a herpesvirus such as a pseudorabies virus (PRV). For PRV, the insertion sites may be in particular in the thymidine kinase gene, in the gE gene, or in the UL43 ORF (see WO 96/13575, WO 87/04463). In one embodiment the polynucleotide to be expressed is inserted under the control of a promoter functional in eukaryotic cells, advantageously a CMV-IE promoter (murine or human). In one particular embodiment a promoter regulated by hypoxia, e.g. the promoter HRE described in K. Boast et al Human Gene Therapy 1999, 13, 2197-2208), can be used. A poly(A) sequence and terminator sequence can be inserted downstream the polynucleotide to be expressed, e.g. bovine growth hormone or a rabbit .beta.-globin gene polyadenylation signal.

According to a yet further embodiment of the invention, the expression vector is a plasmid vector or a DNA plasmid vector, in particular an in vivo expression vector. In a specific, non-limiting example, the pVR1020 or 1012 plasmid (VICAL Inc.; Luke C. et al., Journal of Infectious Diseases, 1997, 175, 91-97; Hartikka J. et al., Human Gene Therapy, 1996, 7, 1205-1217, see, e.g., U.S. Pat. Nos. 5,846,946 and 6,451,769) can be utilized as a vector for the insertion of a polynucleotide sequence. The pVR1020 plasmid is derived from pVR1012 and contains the human tPA signal sequence. In one embodiment the human tPA signal comprises from amino acid M(1) to amino acid S(23) in Genbank under the accession number HUMTPA14. In another specific, non-limiting example, the plasmid utilized as a vector for the insertion of a polynucleotide sequence can contain the signal peptide sequence of equine IGF1 from amino acid M(24) to amino acid A(48) in Genbank under the accession number U28070. Additional information on DNA plasmids which may be consulted or employed in the practice are found, for example, in U.S. Pat. Nos. 6,852,705; 6,818,628; 6,586,412; 6,576,243; 6,558,674; 6,464,984; 6,451,770; 6,376,473 and 6,221,362.

The term plasmid covers any DNA transcription unit comprising a polynucleotide according to the invention and the elements necessary for its in vivo expression in a cell or cells of the desired host or target; and, in this regard, it is noted that a supercoiled or non-supercoiled, circular plasmid, as well as a linear form, are intended to be within the scope of the invention.

Each plasmid comprises or contains or consists essentially of, in addition to the polynucleotide encoding the M. hyopneumoniae immunogen or a variant, analog or fragment thereof, operably linked to a promoter or under the control of a promoter or dependent upon a promoter. In general, it is advantageous to employ a strong promoter functional in eukaryotic cells. The preferred strong promoter is the immediate early cytomegalovirus promoter (CMV-IE) of human or murine origin, or optionally having another origin such as the rat or guinea pig. The CMV-IE promoter can comprise the actual promoter part, which may or may not be associated with the enhancer part. Reference can be made to EP-A-260 148, EP-A-323 597, U.S. Pat. Nos. 5,168,062, 5,385,839, and 4,968,615, as well as to PCT Application No WO87/03905. The CMV-IE promoter is advantageously a human CMV-IE (Boshart M. et al., Cell., 1985, 41, 521-530) or murine CMV-IE.

In more general terms, the promoter has either a viral or a cellular origin. A strong viral promoter other than CMV-IE that may be usefully employed in the practice of the invention is the early/late promoter of the SV40 virus or the LTR promoter of the Rous sarcoma virus. A strong cellular promoter that may be usefully employed in the practice of the invention is the promoter of a gene of the cytoskeleton, such as e.g. the desmin promoter (Kwissa M. et al., Vaccine, 2000, 18, 2337-2344), or the actin promoter (Miyazaki J. et al., Gene, 1989, 79, 269-277).

Functional sub fragments of these promoters, i.e., portions of these promoters that maintain an adequate promoting activity, are included within the present invention, e.g. truncated CMV-IE promoters according to PCT Application No. WO98/00166 or U.S. Pat. No. 6,156,567 can be used in the practice of the invention. A promoter in the practice of the invention consequently includes derivatives and sub fragments of a full-length promoter that maintain an adequate promoting activity and hence function as a promoter, preferably promoting activity substantially similar to that of the actual or full-length promoter from which the derivative or sub fragment is derived, e.g., akin to the activity of the truncated CMV-IE promoters of U.S. Pat. No. 6,156,567 to the activity of full-length CMV-IE promoters. Thus, a CMV-IE promoter in the practice of the invention can comprise or consist essentially of or consist of the promoter portion of the full-length promoter and/or the enhancer portion of the full-length promoter, as well as derivatives and sub fragments.

Preferably, the plasmids comprise or consist essentially of other expression control elements. It is particularly advantageous to incorporate stabilizing sequence(s), e.g., intron sequence(s), preferably the first intron of the hCMV-IE (PCT Application No. WO89/01036), the intron II of the rabbit b-globin gene (van Ooyen et al., Science, 1979, 206, 337-344).

As to the polyadenylation signal (polyA) for the plasmids and viral vectors other than poxviruses, use can more be made of the poly(A) signal of the bovine growth hormone (bGH) gene (see U.S. Pat. No. 5,122,458), or the poly(A) signal of the rabbit beta-globin gene or the poly(A) signal of the SV40 virus.

According to another embodiment of the invention, the expression vectors are expression vectors used for the in vitro expression of proteins in an appropriate cell system. The expressed proteins can be harvested in or from the culture supernatant after, or not after secretion (if there is no secretion a cell lysis typically occurs or is performed), optionally concentrated by concentration methods such as ultrafiltration and/or purified by purification means, such as affinity, ion exchange or gel filtration-type chromatography methods.

Host cells that can be used in the present invention include, but are not limited to, muscle cells, keratinocytes, myoblasts, Chinese Hamster ovary cells (CHO), vero cells, BHK21, sf9 cells, and the like. It is understood to one of skill in the art that conditions for culturing a host cell varies according to the particular gene and that routine experimentation is necessary at times to determine the optimal conditions for culturing M. hyopneumoniae depending on the host cell.

A “host cell” denotes a prokaryotic or eukaryotic cell that has been genetically altered, or is capable of being genetically altered by administration of an exogenous polynucleotide, such as a recombinant plasmid or vector. When referring to genetically altered cells, the term refers both to the originally altered cell and to the progeny thereof.

Polynucleotides comprising a desired sequence can be inserted into a suitable cloning or expression vector, and the vector in turn can be introduced into a suitable host cell for replication and amplification. Polynucleotides can be introduced into host cells by any means known in the art. The vectors containing the polynucleotides of interest can be introduced into the host cell by any of a number of appropriate means, including direct uptake, endocytosis, transfection, f-mating, electroporation, transfection employing calcium chloride, rubidium chloride, calcium phosphate, DEAE-dextran, or other substances; microprojectile bombardment; lipofection; and infection (where the vector is infectious, for instance, a retroviral vector). The choice of introducing vectors or polynucleotides will often depend on features of the host cell.

In an advantageous embodiment, the invention provides for the administration of a therapeutically effective amount of a formulation for the delivery and expression of a M. hyopneumoniae immunogen in a target cell. Determination of the therapeutically effective amount is routine experimentation for one of ordinary skill in the art. In one embodiment, the formulation comprises an expression vector comprising a polynucleotide that expresses a M. hyopneumoniae immunogen and a pharmaceutically or veterinarily acceptable carrier, vehicle or excipient. In an advantageous embodiment, the pharmaceutically or veterinarily acceptable carrier, vehicle or excipient facilitates transfection and/or improves preservation of the vector or protein.

The pharmaceutically or veterinarily acceptable carriers or vehicles or excipients are well known to the one skilled in the art. For example, a pharmaceutically or veterinarily acceptable carrier or vehicle or excipient can be a 0.9% NaCl (e.g., saline) solution or a phosphate buffer. Other pharmaceutically or veterinarily acceptable carrier or vehicle or excipients that can be used for methods of this invention include, but are not limited to, poly-(L-glutamate) or polyvinylpyrrolidone. The pharmaceutically or veterinarily acceptable carrier or vehicle or excipients may be any compound or combination of compounds facilitating the administration of the vector (or protein expressed from an inventive vector in vitro); advantageously, the carrier, vehicle or excipient may facilitate transfection and/or improve preservation of the vector (or protein). Doses and dose volumes are herein discussed in the general description and can also be determined by the skilled artisan from this disclosure read in conjunction with the knowledge in the art, without any undue experimentation.

The cationic lipids containing a quaternary ammonium salt which are advantageously but not exclusively suitable for plasmids, are advantageously those having the following formula: 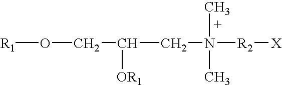
in which R₁ is a saturated or unsaturated straight-chain aliphatic radical having 12 to 18 carbon atoms, R₂ is another aliphatic radical containing 2 or 3 carbon atoms and X is an amine or hydroxyl group, e.g. the DMRIE. In another embodiment the cationic lipid can be associated with a neutral lipid, e.g. the DOPE.

Among these cationic lipids, preference is given to DMRIE (N-(2-hydroxyethyl)-N,N-dimethyl-2,3-bis(tetradecyloxy)-1-propane ammonium; WO96/34109), advantageously associated with a neutral lipid, advantageously DOPE (dioleoyl-phosphatidyl-ethanol amine; Behr J. P., 1994, Bioconjugate Chemistry, 5, 382-389), to form DMRIE-DOPE.

Advantageously, the plasmid mixture with the adjuvant is formed extemporaneously and advantageously contemporaneously with administration of the preparation or shortly before administration of the preparation; for instance, shortly before or prior to administration, the plasmid-adjuvant mixture is formed, advantageously so as to give enough time prior to administration for the mixture to form a complex, e.g. between about 10 and about 60 minutes prior to administration, such as approximately 30 minutes prior to administration.

When DOPE is present, the DMRIE:DOPE molar ratio is advantageously about 95: about 5 to about 5:about 95, more advantageously about 1: about 1, e.g., 1:1.

The immunogenic compositions and vaccines according to the invention advantageously comprise or consist essentially of one or more adjuvants. Suitable adjuvants for use in the practice of the present invention are (1) polymers of acrylic or methacrylic acid, maleic anhydride and alkenyl derivative polymers, (2) immunostimulating sequences (ISS), such as oligodeoxyribonucleotide sequences having one ore more non-methylated CpG units (Klinman D. M. et al., Proc. Natl. Acad. Sci., USA, 1996, 93, 2879-2883; W098/16247), (3) an oil in water emulsion, such as the SPT emulsion described on p 147 of “Vaccine Design, The Subunit and Adjuvant Approach” published by M. Powell, M. Newman, Plenum Press 1995, and the emulsion MF59 described on p 183 of the same work, (4) cation lipids containing a quaternary ammonium salt, (5) cytokines, (6) aluminum hydroxide or aluminum phosphate or (7) saponin, (8) Dimethyldioctadecyl ammonium bromide (Vaccine Design p. 157), (9) Aridine (Vaccine Design p. 148) other adjuvants discussed in any document cited and incorporated by reference into the instant application, or (8) any combinations or mixtures thereof.

The oil in water emulsion (3), which is especially appropriate for viral vectors, can be based on: light liquid paraffin oil (European pharmacopoeia type), isoprenoid oil such as squalane, squalene, oil resulting from the oligomerization of alkenes, e.g. isobutene or decene, esters of acids or alcohols having a straight-chain alkyl group, such as vegetable oils, ethyl oleate, propylene glycol, di(caprylate/caprate), glycerol tri(caprylate/caprate) and propylene glycol dioleate, or esters of branched, fatty alcohols or acids, especially isostearic acid esters. The oil is used in combination with emulsifiers to form an emulsion. The emulsifiers may be nonionic surfactants, such as: esters of on the one hand sorbitan, mannide (e.g. anhydromannitol oleate), glycerol, polyglycerol or propylene glycol and on the other hand oleic, isostearic, ricinoleic or hydroxystearic acids, said esters being optionally ethoxylated, or polyoxypropylene-polyoxyethylene copolymer blocks, such as Pluronic, e.g., L121.

The polymers of acrylic or methacrylic acid (1) are preferably crosslinked, in particular with polyalkenyl ethers of sugars or polyalcohols. These compounds are known under the term carbomer (Pharmeuropa vol. 8, No. 2, June 1996). Persons skilled in the art can also refer to U.S. Pat. No. 2,909,462 describing such acrylic polymers crosslinked with a polyhydroxylated compound having at least 3 hydroxyl groups, preferably not more than 8, the hydrogen atoms of at least three hydroxyls being replaced with unsaturated aliphatic radicals having at least 2 carbon atoms. The preferred radicals are those containing 2 to 4 carbon atoms, e.g. vinyls, allyls and other ethylenically unsaturated groups. The unsaturated radicals may themselves contain other substituents, such as methyl. The products sold under the name Carbopol™ (BF Goodrich, Ohio, USA) are particularly appropriate. They are crosslinked with an allyl sucrose or with allylpentaerythritol. Among them, there may be mentioned Carbopol™ 974P, 934P and 971 P.

Among the copolymers of maleic anhydride and of alkenyl derivative, the EMA™ copolymers (Monsanto) which are copolymers of maleic anhydride and of ethylene, which are linear or crosslinked, for example crosslinked with divinyl ether, are preferred. Reference may be made to J. Fields et al., Nature, 186: 778-780, Jun. 4, 1960.

The proportions of adjuvant which are useful are well known and readily available to the one skilled in the art. By way of example, the concentration of polymers of acrylic or methacrylic acid or of anhydride maleic and alkenyl copolymers in the final vaccine composition will be from 0.01% to 1.5% W/V, more particularly from 0.05 to 1% W/v, preferably from 0.1 to 0.4% W/V.

In an advantageous embodiment, the adjuvant is an adjuvant disclosed in U.S. patent application Ser. No. 10/899,181 filed Jul. 26, 2004 and published as U.S. patent publication No. 2005/0079185 on Apr. 14, 2005. In an advantageous embodiment, the adjuvant is a TS6 adjuvant.

Optionally the vaccine used according to the method of the invention may contain a cytokine. The cytokine may be present as a protein or as a gene encoding this cytokine inserted into a recombinant viral vector. The cytokines may be selected among the feline cytokines, e.g. feline interleukine 18 (flL-18) (Taylor S. et al., DNA Seq., 2000, 10(6), 387-394), flL-16 (Leutenegger C. M. et al., DNA Seq., 1998, 9(1), 59-63), flL-12 (Fehr D. et al., DNA Seq., 1997, 8(1-2), 77-82; Imamura T. et al., J. Vet. Med. Sci., 2000, 62(10), 1079-1087) and feline GM-CSF (Granulocyte-Macrophage Colony-Stimulating Factor) (GenBank AF053007).

In a specific embodiment, the pharmaceutical composition is directly administered in vivo, and the encoded product is expressed by the vector in the host. Advantageously, the pharmaceutical and/or therapeutic compositions and/or formulations according to the invention comprise or consist essentially of or consist of an effective quantity to elicit a therapeutic response of one or more expression vectors and/or polypeptides as discussed herein; and, an effective quantity can be determined from this disclosure, including the documents incorporated herein, and the knowledge in the art, without undue experimentation.

In the case of therapeutic and/or pharmaceutical compositions based on a plasmid vector, a dose can comprise, consist essentially of or consist of, in general terms, about in 1 μg to about 2000 μg, advantageously about 50 μg to about 1000 μg and more advantageously from about 100 μg to about 800 μg of plasmid expressing a M. hyopneumoniae immunogen. When the therapeutic and/or pharmaceutical compositions based on a plasmid vector is administered with electroporation the dose of plasmid is generally between about 0.1 μg and 1 mg, advantageously between about 1 pg and 100 μg, advantageously between about 2 μg and 50 μg. The therapeutic and/or pharmaceutical composition contains per dose from about 10⁴ to about 10¹¹, advantageously from about 10⁵ to about 10¹⁰ and more advantageously from about 10⁶ to about 10⁹ viral particles of recombinant adenovirus expressing a M. hyopneumoniae immunogen. In the case of therapeutic and/or pharmaceutical compositions based on a poxvirus, a dose can be between about 10² pfu and about 10⁹ pfu. The pharmaceutical composition contains per dose from about 10⁵ to 10⁹, advantageously from about 10⁶ to 10⁸ pfu of poxvirus or herpesvirus recombinant expressing a M. hyopneumoniae immunogen.

It should be understood by one of skill in the art that the disclosure herein is provided by way of example and the present invention is not limited thereto. From the disclosure herein and the knowledge in the art, the skilled artisan can determine the number of administrations, the administration route, and the doses to be used for each injection protocol, without any undue experimentation.

The present invention contemplates at least one administration to an animal of an efficient amount of the therapeutic composition made according to the invention. The animal may be male, female, pregnant female and newborn. This administration may be via various routes including, but not limited to, intramuscular (IM), intradermal (ID) or subcutaneous (SC) injection or via intranasal or oral administration. The therapeutic composition according to the invention can also be administered by a needleless apparatus (as, for example with a Pigjet, Biojector or Vitajet apparatus (Bioject, Oreg., USA)). Another approach to administer plasmid compositions is to use electroporation (see, e.g. S. Tollefsen et al. Vaccine, 2002, 20, 3370-3378; S. Tollefsen et al. Scand. J. Immunol., 2003, 57, 229-238; S. Babiuk et al., Vaccine, 2002, 20, 3399-3408; PCT Application No. WO 99/01158). In another embodiment, the plasmid is delivered to the animal by gene gun or gold particle bombardment. In an advantageous embodiment, the animal is a pig.

Advantageously, the M. hyopneumoniae vaccine is administered to a weaned pig (about two to three weeks old). The pig may be about 10, about 11, about 12, about 13, about 14, about 15, about 16, about 17, about 18, about 19 days, about 20, about 21, about 22, about 23, about 24 days of age. A booster administration can be done if necessary around 3 to 5 weeks after the first administration. In another advantageous embodiment, the M. hyopneumoniae vaccine of the present invention is administered to a sow so that piglets acquire immunity against M. hyopneumoniae. The administration is dermal (epidermis and dermis) or subdermal (hypodermis and/or subcutaneous and/or intramuscular), advantageously in the neck, more advantageously in the neck region caudal to the ear. This route of administration may allow to target the immunogen to the dendritic Langerhan's cells.

Liquid jet needle-free injectors are devices performing injections of a certain amount of liquid under high pressure through a minute orifice. Mechanical specifications of the injector may be adjusted or selected in order to control the depth of penetration into tissues. Administrations of a liquid using a syringe or a needle-free injector end up in a different distribution of the liquid in the tissues. Using a syringe end up in a localized bolus or pool. Using an injector end up in a diffused distribution in the layers of the targeted tissues, as illustrated in WO-A-01/13975. In an advantageous embodiment, the needle-free injection is a DERMA-VAC NF transdermal vaccinator system.

The depth of penetration is mainly controlled by the liquid pressure. This liquid pressure is depending upon the mechanical specifications of the injector, such as the strength of spring or any other propulsion means and the diameter of the piston and the nozzle orifice. This is readily available to the one skilled in the art.

The depth of injection may be easily determined by the dissection of the tissue at the injection site (corresponding preferably to the location where the vaccine is going to be administered, and the test is advantageously performed on an animal of the same species and age than the population to be vaccinated) after the administration of a colored liquid having preferably the same viscosity than the intended vaccine. This test may be performed directly with the intended vaccine containing further a dye. This test allows the one skilled in the art to adjust the mechanical specifications of an injector.

The needle-free injector may be equipped with a head comprising one or several nozzles. The use of several nozzles allows to increase the dispersion pattern of the vaccine over a larger area. There can be from 1 to 10 nozzles, preferably from 1 to 6.

Several injectors are available in the commerce. The Vitajet™ 3 (Bioject Inc.) is particularly adapted to the method according to the invention.

It is advantageous to use an injector equipped with means allowing to fit to the injector directly a standard vial or ampoule. In addition, the vaccine vial may comprise several vaccine doses allowing several shots of vaccine and/or vaccination of several animals using the injector and the same vial. Thus, the injector is preferably able to perform successive injections from a same vial.

In one aspect of the invention, vaccination against M. hyopneumoniae can be associated with a vaccination against another porcine disease. The vaccine comprises the M. hyopneumoniae vaccine according to the invention and a vaccine component able to protect against other porcine disease The volume of dose injected may be from about 0.1 ml to about 1.0 ml, preferably about 0.1 ml to about 0.8 ml, more preferably from about 0.2 ml to about 0.5 ml. By definition, the volume of one dose means the total volume of vaccine administered at once to one animal.

The vaccine may contain from about 10^{4.5} to about 10^{8.0} TCID₅₀/dose (50% tissue culture infective dose per dose of vaccine) and preferably from about 10^{5.5} to about 10^{6.5} TCID₅₀/dose.

Optionally, the administration can be repeated, as booster administration, at suitable intervals if necessary or desirable, e.g. about from 2 to about 8 weeks after the first administration, and preferably about from 3 to about 5 weeks after the first administration. A booster administration can also be repeated every year, especially for the sows.

Another object of the invention is the use of an efficient amount of a M. hyopneumoniae vaccine as described above and of an acceptable vehicle or diluent, for the preparation of a vaccine designed to be administered to an animal using a liquid jet needle-free injector as described above, and resulting in eliciting a safe and protective immune response against M. hyopneumoniae.

Another object is a vaccination kit or set, comprising such a liquid jet needle-free injector and at least one vaccine vial containing a M. hyopneumoniae vaccine, operatively assembled to perform the administration of the vaccine to an animal of the porcine family. The distribution of the vaccine is essentially done in the dermis and the hypodermis.

Such vaccination kit or set is able to elicit a safe and protective immune response against M. hyopneumoniae.

The invention will now be further described by way of the following non-limiting examples.

### EXAMPLE 1

### Efficacy and Safety of Needle-Free Transdermal Delivery of a Novel Mycoplasma hyopneumoniae Bacterin

Mycoplasma hyopneumoniae remains among the leading agents of respiratory diseases in swine. Vaccination is now part of standard management practices globally. Any improvements that will increase the overall efficiency of vaccination programs are very desirable. These would include improved vaccine efficacy through new formulations and antigen presentations, improved compliance, improved safety for the animal and the vaccinator, and elimination of the risk of needles in the final product (see, e.g., Almond G. W. and J. D. Roberts, 2004, Assessment of a Needleless Injection Device for Iron Dextran administration to piglets, Proceedings of the IPVS, 618; Houser T. A., J. G. Sebranek, T. J. Baas, B. J. Thacker, D. Nilubol and E. L. Thacker, 2002, Feasibility of Transdermal, Needleless Injections for Prevention of Pork Carcass Defects http://www.ipic.iastate.edu/reports/02swinereports/asl-1814.pdf; Meredith M. J., 2004, AASV New archive, Needle-Free Vaccination http://www.aasp.org/news/stroy.php?id=1279; Sweat J. M., M. Abdy, B. G. Weniger, R. Harrington, B. Coyle, R. A. Abuknesha and E. P. J. Gibbs, 2000, Safety Testing of Needle Free, Jet Injection Devices to Detect Contamination with Blood and Other Tissue Fluids, Annals of the New York Academy of Sciences 916:681-682 and Willson P., 2004, Council Research News, Needle-Free Immunization as Effective as Needle and Syringe Method http://www.manitobapork.com/admin/docs/research_news_ly.pdf).

The long-term efficacy and safety of a novel formulation specifically designed to be administered transdermally using the DERMA-VAC™ NF Transdermal Vaccinator System is presented in this Example. Needle-free administration has the potential to address both safety and quality aspects of these objectives as well as to provide an optimized presentation of the vaccine to the immune system (see, e.g., Charreyre C., F. Milward, R. Nordgren and G. Royer, 2005, Demonstration of efficacy in pigs of Mycoplasma hyopneumoniae experimental vaccines by an innovative needle-free route, Proceedings of the American Association of Swine Veterinarians).

Forty-eight conventional pigs, sixteen to nineteen days of age, were obtained from conventional dams originating in a low Mycoplasma hyopneumoniae incidence herd.

Vaccinations were performed with the DERMA-VAC NF Transdermal Vaccinator System. This device uses compressed air to deliver 0.5 ml of a specially formulated bacterin. Pigs were injected in the neck region caudal to the ear with Mycoplasma hyopneumoniae bacterin formulated for needle-free administration. The vaccine was administered the same day the animals arrived at the trial site (day 0).

Pigs were penned by replicate with all members of the replicate consisting of littermates. Pigs were randomized to treatment group within replicate so that each treatment group was equally represented in the replicate:
Group 1 (24 pigs) consisted of unvaccinated controls.
Group 2 (24 pigs) was vaccinated by the transdermal route.

Pigs were housed in environmentally controlled buildings which were appropriate for their age and size. From day 0 until day 21, pigs were housed within a pre-nursery on elevated, plastic slotted commercial swine flooring in solid-sided pens. From day 21 until day 51 pigs were housed in a conventional nursery. The nursery facility utilized plastic slotted commercial swine flooring over a pit with galvanized metal barred gate penning. From day 51 through necropsy (day 189, 190 or 191) pigs were housed in a finishing building which utilized concrete slats over a pit with galvanized metal barred gate penning. Replicate penning was maintained throughout the study. Feed and water were available ad libitum.

Blood samples were collected on day −2 (offsite), days 21, 42, 125, 155 (pre-challenge), and on days 189, 190 or 191 (the day of necropsy). Sera from blood samples were tested for determination of Mycoplasma hyopneumoniae ELISA antibody titers.

On days 160 and 161, all pigs were challenged intranasally with 25 ml of Mycoplasma hyopneumoniae lung homogenate at a dilution 1/100 with Friis (Mycoplasma hyopneumoniae strain 232). On day 162, pigs received 20 ml of a previously frozen culture of Mycoplasma hyopneumoniae strain SC2 via the intranasal route. Challenge material was administered into both nostril openings with a syringe in a manually restrained, alert pig.

At necropsy, lungs were removed, photographed and scored for percentage of pneumonic tissue characteristic of Mycoplasma hyopneumoniae. The lungs were independently scored by two different individuals who were blinded to treatment assignment. Specifically, the total percentage of lung affected with lesions was determined for each animal. The percentage of pneumonic tissue was estimated by visual observation of the dorsal and ventral surfaces of the cranial, middle, and caudal lobes and all visible surfaces of the accessory lobe. The calculation of the percentage of lesions in pneumonic tissue was based upon the percentage of total lung mass represented by each lobe as indicated in Table 1.

TABLE 1
Percentage of total lung mass represented by each lobe for the
calculation of the percent of lesions in pneumonic tissue.

| | |
|---|---|
| Left cranial | 7% |
| Left caudal | 30% |
| Right middle | 7% |
| Accessory | 5% |
| Left middle | 7% |
| Right cranial | 12% |
| Right caudal | 32% |

In a separate field safety study, six-hundred-sixty-three pigs at commercial sites located in three different geographical regions (MO, PA, NC) of the US were vaccinated with one of two different pre-license serials of Merial's Mycoplasma hyopneumoniae bacterin formulated for needle-free administration. The bacterin was administered using the DERMA-VAC NF Transdermal Vaccinator System. Pigs were observed for adverse systemic events post-injection. Injection sites were observed visually and by palpation on days 1, 3, 7, 14 and 21 in all pigs and days 34 or 35 in pigs with persistent injection site swellings.

In the efficacy study, one pig from Group 1 was removed on day 162 subsequent to an injury. Lung scores from one additional pig in Group 1 were not included in the analysis due to the presence of excessive adhesions. The final group sizes were:
Group 1, Unvaccinated controls, 23 pigs (serology), 22 pigs (lung scores)
Group 2, Test vaccine transdermal, 24 pigs

Average ELISA titers to Mycoplasma hyopneumoniae antibodies remained at values considered negative until after challenge in the control group. Test vaccinated pigs had seroconverted to antibody positive status by day 21 post-vaccination, remained positive through day 125, dipping into suspect status at the day 155 bleed and showing a substantial anamnestic response post-challenge (Table 2).

TABLE 2
Average Mycoplasma hyopneumoniae
ELISA antibody titers over time.

| | D −2 | D 21 | D 42 | D 125 | D 155 | D 189-191 |
|---|---|---|---|---|---|---|
| Group 1 | <0.001 | 0.03 | 0.05 | 0.11 | 0.18 | 0.64 |
| Controls | | | | | | |
| Group 2 | 0.000 | 0.45 | 0.57 | 0.51 | 0.33 | 1.30 |
| Test | | | | | | |
| Vaccine | | | | | | |

The averaged percentage of pneumonic tissue in the control group was 4.35%, versus 1.72% for the vaccinated group (Table 3). Pigs vaccinated with the Mycoplasma hyopneumoniae bacterin administered via the transdermal route, exhibited a significantly lower (p<0.05) percentage of pneumonic lung lesions than were observed in the unvaccinated controls. During the field safety study, no systemic adverse events associated with vaccination were observed. Palpable injection site swellings were typical of adjuvanted bacterins and dropped to negligible levels by day 35 post-vaccination.

TABLE 3
Mean Percentage and Standard Deviation
of pneumonic tissue in lungs.

| Group | Vaccine | N | Mean %* | SD |
|---|---|---|---|---|
| 1 | Unvaccinated | 22 | 4.35 | 3.93 |
| | Controls | | | |
| 2 | Test Vaccine | 24 | 1.72 | 1.82 |

*(p < 0.05)

The Mycoplasma hyopneumoniae bacterin administered using the DERMA-VAC NF Transdermal Vaccinator System protected pigs safely and significantly against Mycoplasma hyopneumoniae challenge 160 days after a single injection of vaccine administered before 3 weeks of age. The overall safety of the Mycoplasma hyopneumoniae bacterin administered with the DERMA-VAC NF Transdermal Vaccinator System was also demonstrated in field conditions in a large number of animals. The new DERMA-VAC NF Transdermal Vaccinator System provides a unique presentation of a Mycoplasma hyopneumoniae vaccine specially formulated for needle-free use, achieving a safe, convenient and efficacious vaccination protocol.

### EXAMPLE 2

A mycoplasma hyopneumoniae bacterin formulated with a TS6 oil-in-water emulsion according to US Pat. Appl. No. 2005/0079185, at different doses of immunogen determined by ELISA, was administered to 16-19 day-old piglets either by intramuscular route (IM) with a syringe and a needle (2 ml) or by transdermal route (ID) with a needle-less device (0.5 ml) in the neck region. The needle-less injector was a DERMA-VAC NF transdermal vaccinator system with a nozzle #3. The pigs were challenged 35 days post immunization by intratracheal administration of 10 ml of a Mycoplasma hyopneumoniae (strain 232) lung homogenate at a dilution 1/100 with Friis medium and and at 36 days post immunization by administration of 10 ml of the same suspension by the intranasal route. 61-62 days after immunization the pigs were necropsied and the lung lesions were scored as described in Example 1.

| Vaccine | Route | N | Average percent |
|---|---|---|---|
| Unvaccinated control | | 19 | 11.23 |
| Vaccine 1: 6 Ag units/2 ml dose | IM | 19 | 1.04 |
| Vaccine 2: 1.5 Ag units/0.5 ml | ID (NF) | 20 | 3.24 |
| Vaccine 3: 3 Ag units/0.5 ml | ID (NF) | 19 | 2.49 |

In the groups corresponding to each of vaccines 1-3, the percent of pneumonic lung tissue was significantly decreased (p<0.0001) in comparison to the control group. The lesion scores in vaccinated groups 1, 2 and 3 were not different. The use of a needle-less injector allowed for a ¼ reduction in the dose of immunogen in the vaccine.

The invention is further described by the following numbered paragraphs:

1. A method of eliciting a safe and protective immune response against Mycoplasma hyopneumoniae comprising administering a single dose of a vaccine comprising an inactivated Mycoplasma hyopneumoniae and an adjuvant, to porcines with a liquid jet needle-free injector.

2. The method according to paragraph 1, wherein the adjuvant is a TS6 adjuvant.

3. The method according to paragraph 1 or 2, wherein the porcine is a weaned piglet.

4. The method according to paragraph 3, wherein the weaned piglet is about 11 to about 24 days of age.

5. The method according to paragraph 3, wherein the weaned piglet is about two to three weeks of age.

6. The method according to any one of paragraphs 1 to 5, wherein the porcine is a sow.

7. The method according to any one of paragraphs 1 to 5, wherein the needle-free injector is a DERMA-VAC NF injector.

8. A vaccination kit or set, comprising a liquid jet needle-free injector and at least one vaccine vial containing Mycoplasma hyopneumoniae vaccine, operatively assembled to perform the administration of the vaccine to a porcine and to elicit a safe and protective immune response against Mycoplasma hyopneumoniae.

9. The vaccination kit or set of paragraph 8 comprising the compositions of any one of paragraphs 1 to 7 and instructions for performing any one of the methods of paragraphs 1 to 7.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A method of eliciting a safe and protective immune response against Mycoplasma hyopneumoniae comprising administering a single dose of a vaccine comprising an inactivated Mycoplasma hyopneumoniae and an adjuvant, to porcines with a liquid jet needle-free injector.

2. The method according to claim 1, wherein the adjuvant is a TS6 adjuvant.

3. The method according to claim 1, wherein the porcine is a weaned piglet.

4. The method according to claim 2, wherein the porcine is a weaned piglet.

5. The method according to claim 3, wherein the porcine is a weaned piglet.

6. The method according to claim 4, wherein the weaned piglet is about 11 to about 24 days of age.

7. The method according to claim 5, wherein the weaned piglet is about 11 to about 24 days of age.

8. The method according to claim 4, wherein the weaned piglet is about two to three weeks of age.

9. The method according to claim 5, wherein the weaned piglet is about two to three weeks of age.

10. The method according to claim 1, wherein the porcine is a sow.

11. The method according to claim 1, wherein the needle-free injector is a DERMA-VAC NF injector.

12. A vaccination kit or set, comprising a liquid jet needle-free injector and at least one vaccine vial containing Mycoplasma hyopneumoniae vaccine, operatively assembled to perform the administration of the vaccine to a porcine and to elicit a safe and protective immune response against Mycoplasma hyopneumoniae.

13. The vaccination kit or set of claim 12, wherein the adjuvant is a TS6 adjuvant.

14. The vaccination kit or set of claim 12, wherein the needle-free injector is a DERMA-VAC NF injector.

15. The vaccination kit or set of claim 13, wherein the needle-free injector is a DERMA-VAC NF injector.
